(19) United States
(12) Patent Application Publication
(10) Pub. No.: US 2004/0142937 A1
(43) Pub. Date: Jul. 22, 2004

(54) Use of heterocyclic amine-type compounds as neuroprotective agents

(76) Inventors: Haiyan Wu, Eugene, OR (US); Jo Ann Oostveen, Galesburg, MI (US); Vimala Hiralal Sethy, Kalamazoo, MI (US); Edward Dallas Hall, Lexington, KY (US); Robert Bradford McCall, Kalamazoo, MI (US)

Correspondence Address: PHARMACIA & UPJOHN, 301 HENRIETTA ST, 0228-32-LAW, KALAMAZOO, MI, 49007, US

(21) Appl. No.: 10/690,010

(22) Filed: Oct. 21, 2003

Related U.S. Application Data
(60) Provisional application No. 60/421,352, filed on Oct. 25, 2002.

(51) Int. Cl. A61K 31/5377; A61K 31/496; A61K 31/519

(52) U.S. Cl. 514/253.03; 514/267; 514/234.2; 514/294

(57) ABSTRACT

The present invention provides the use of heterocyclic amine-type compounds to prevent or reduce neuronal damages in patients afflicted with or susceptible to disease states or conditions known to result in or cause neuronal damage.

### CROSS REFERENCE

This application claims the benefit of the following provisional application: U.S. Ser. No. 60/421,352, filed 25 Oct. 2002 under 35 USC 119(e)(i), which is incorporated herein by reference in its entirety.

### FIELD OF THE INVENTION

The present invention relates to a method of treatment for preventing or reducing neuronal damages in the central nervous system in subjects.

### BACKGROUND OF THE INVENTION

Many pathological conditions or disorders are known to result from or in loss of neuronal cells or loss or neuronal functions in the central nervous system, including acute or chronic neurodegenerative diseases. Example of a chronic neurodegenerative diseases include Alzheimer's disease, Parkinson's disease; Huntington's disease; AIDS Dementia; Wernicke-Korsakoff's related dementia (alcohol induced dementia); age related dementia; age associated memory impairment; brain cell loss due to head trauma, stroke, hypoglycemia, ischemia, anoxia, hypoxia, cerebral edema, arteriosclerosis, hematoma or epilepsy; spinal cord cell loss due to any of the conditions listed under brain cell loss; and peripheral neuropathy. Other conditions known to result in loss of neuronal cells or loss of neuronal cell function are those generally characterized as secondary neurodegenerative disease of typically metabolic or toxic origin.

Chronic and acute neurodegenerative diseases and acute nerve cell injury, as well as associated mortality and morbidity, have been largely untreatable with previous methods. Patient disability resulting from these conditions can cause a significant reduction in quality of life. In addition, these conditions impose a high cost to the patient and to society for long-term care. Accordingly, effective therapeutic approaches directed to the prevention or reduction of nerve cell death or nerve cell damage associated with neurodegenerative diseases and acute nerve cell injury are needed. Specifically, an efficacious method for treating conditions in the brain resulting from neuron loss is needed that is relatively non-toxic and that can readily access the brain across the blood-brain barrier.

Surprisingly and unexpectedly, it has been found that sumanirole and its analogues are advantageously suitable as a treatment for prevention or reduction of nerve cell death or nerve cell damage. Compounds of the invention not only have neuprotective effect, they also have also been shown to have remarkable safety profile and to readily penetrate the brain-blood barrier.

### INFORMATION DISCLOSURE

U.S. Pat. No. 6,458,820 discloses pramipexole, a dopamine receptor agonist, as a neuroprotective agent.

U.S. Pat. Nos. 5,273,975 and 5,436,240, and in International Patent Application WO 00/40226 disclose the compounds of the invention useful for treating symptoms of Parkinson diseases.

U.S. Pat. No. 6,426,342 discloses a method to prevent or reduce loss of neuronal cells and neuronal cell function in patients using beta-Lactamase inhibitors.

U.S. Pat. No. 6,451,837 discloses a method of protecting nerve cells from deterioration and cell death with a natural or synthetic bioflavonoid that acts as an MAPK cascade antagonist.

Piribedil, a vasodilator which binds to a multitude of receptors including dopamine receptors, is reported to have an effect on functional and biochemical parameters in a gerbil model of global cerebral ischemia. See, e.g., Society for Neuroscience Abstracts, 19:673 (1993); id., at 1645.

Lisuride binds to several different receptors including dopamine D₂ and 5-HT1a receptors. It is reported that Lisuride, when administered before the event, reduced brain edema and prolonged survival time in a rat model of cerebral infarction. Miya Zawa, et al. Nippon-Yakurigaku-Zasshi 98(6):449-561, (1991).

### SUMMARY OF THE INVENTION

Disclosed is a method of preventing or reducing neuronal damage or the progression of neuronal damage in a human suffering from or susceptible to disease states causing such neuronal damage, which method comprises administering to the human a neuroprotective amount of a compound of formula (A),

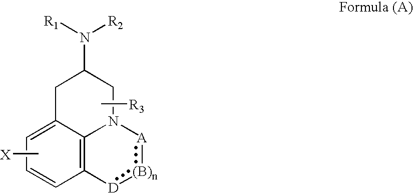

or a pharmaceutically acceptable salt thereof, wherein:

R₁, R₂, and R₃ are independently hydrogen, C₁₋₆ alkyl, C₃₋₅ alkenyl, C₃₋₅ alkynyl, C₃₋₇ cycloalkyl, C_{4 10} cycloalkyl- or phenyl-substituted C₁₋₆ alkyl, or R₁ and R₂ are joined to form a C₃₋₇ cyclic amine which can contain additional heteroatoms and/or unsaturation;

X is hydrogen, C₁₋₆ alkyl, halogen, hydroxy, alkoxy, cyano, carboxamide, carboxyl, or carboalkoxyl;

A is CH, CH₂, CH-halogen, CHCH₃, C═O, C═S, C—SCH₃, C═NH, C—NH₂, C—NHCH₃, C—NHCOOCH₃, C—NHCN, SO₂, or N;

B is CH₂, CH, CH-halogen, C═O, N, NH or N—CH₃, or O;

n is 0 or 1; and

D is CH, CH₂, CH-halogen, C═O, O, N, NH, or N—CH₃.

Also disclosed is a method for the treatment of a patient suffering from or susceptible to a condition known to result in or from loss of neuronal cells or loss of neuronal cell function, which method comprises administering to a patient in need of such treatment a neuroprotective amount of a compound of formula (A) or a pharmaceutically acceptable salt thereof.

Further disclosed is a neuroprotective pharmaceutical composition and a method for manufacturing same comprising a compound of formula (A) or a pharmaceutically acceptable salt thereof as the active ingredient. Such pharmaceutical compositions can be formulated in unit dosage forms adapted for patient delivery by a wide variety of routes of administration including, but not limited to, oral ingestion, buccal, sublingual, parenteral, transdermal and rectal routes of administration. In one embodiment the dosage forms are formulated for controlled release of the active agent.

### DESCRIPTION OF THE PREFERRED EMBODIMENTS

In one aspect, the invention provides a method of preventing or reducing neuronal damage or the progression of neuronal damage in a human suffering from or susceptible to disease states causing such neuronal damage, which method comprises administering to a patient in need of such treatment a neuroprotective amount of a compound of formula (A) or a pharmaceutically acceptable salt thereof. Neuronal damage is characterized generally as a loss of neuronal cells or a loss of neuronal cell function. Examples of disease tates that may cause neuronal damage include strokes, seizures, neural trauma, and a multiplicity of neurodegenerative disease states of widely variant etiology, such as Huntington's Chorea, Parkinson's disease, Alzheimer's disease and other memory disorders, vascular dementia, multi-infarct dementia, Lewy body dementia, or neurogenerative dementia. A particular indication for the compounds of the invention is Parkinson's disease. In this sense, the term Parkinson's disease also comprises the term Parkinson's syndrome.

In another aspect, this invention provides a method for the treatment of a human suffering from or susceptible to a condition known to result in or from loss of neuronal cells or loss of neuronal cell function which method comprises administering the human a neuroprotective amount of a compound of formula (A) or a pharmaceutically acceptable salt thereof.

In still another aspect, the invention provides a neuroprotective pharmaceutical composition comprising as the active ingredient a compound of formula (A) or a pharmaceutically acceptable salt thereof.

In yet another aspect, the invention provides a method for manufacturing a neuroprotective pharmaceutical composition comprising as the active ingredient a compound of formula (A) or a pharmaceutically acceptable salt thereof.

Preferred compounds of formula (A) include the compound of formula (AI) and (AII),

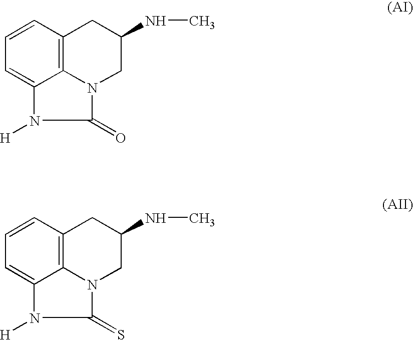

and their pharmaceutically acceptable salts.

A chemical name for the compound of formula (AI) is (R)-5,6-Dihydro-5-(methylamino)-4H-imidazo[4,5,1-ij]-quinolin-2(1H)-one (uninverted CAS name) or (5R)-5-(methylamino)-5,6-dihydro-4H-imidazo[4,5,1-ij]quinolin-2(1H)-one (Generated by ACD/Name software. For the present invention it is preferred that or (5R)-5-(methylamino)-5,6-dihydro-4H-imidazo[4,5,1-ij]quinolin-2(1H)-one be present in a pharmaceutically acceptable salt.

Chemical name of the compound of formula (AII) is (5R)-5-(methylamino)-5,6-dihydro-4H-imidazo[4,5,1-ij]quinoline-2(1H)-thione. It is preferred that (5R)-5-(methylamino)-5,6-dihydro-4H-imidazo[4,5,1-ij]quinoline-2(1H)-thione be present as a pharmaceutically acceptable salt. Pharmaceutically acceptable salts include salts of both inorganic and organic acids.

Suitable pharmaceutically acceptable salts include salts of both inorganic and organic acids; examples include without limitation salts of the following acids: methanesulfonic, hydrochloric, hydrobromic, sulfuric, phosphoric, nitric, benzoic, citric, tartaric, fumaric, maleic, CH₃—(CH₂)ₙ—COOH where n is 0 thru 4, HOOC—(CH₂)_{N}—COOH where n is as defined above. For other acceptable salts, see Int. J. Pharm., 33, 201-217 (1986).

A particularly preferred salt of (5R)-5-(methylamino)-5,6-dihydro-4H-imidazo[4,5,1-ij]quinolin-2(1H)-one and of or (5R)-5-(methylamino)-5,6-dihydro-4H-imidazo[4,5,1-ij]quinolin-2(1H)-thione is the maleate salt. i.e. (Z)-2-butenedioate salt, which is (5R)-5-(methylamino)-5,6-dihydro-4H-imidazo[4,5,1-ij]quinolin-2(1H)-one (Z)-2-butenedioate (1:1) and (5R)-5-(methylamino)-5,6-dihydro-4H-imidazo[4,5,1-ij]quinolin-2(1H)-thione Z)-2-butenedioate (1:1), respectively. (5R)-5-(methylamino)-5,6-dihydro-4H-imidazo[4,5,1-ij]quinolin-2(1H)-one (Z)-2-butenedioate (1:1) is also known by the generic name “sumanirole.”

Compounds of formula (A) and pharmaceutically acceptable salts thereof, which hare useful in the method of the present invention, are known, see, for example, U.S. Pat. Nos. 5,273,975 and 5,436,240, and in International Patent Application WO 00/40226. The full disclosure of the above-cited U.S. Pat. Nos. 5,273,975 and 5,436,240 and International Patent Application WO 00/40226 is incorporated herein by reference. A preferred process of making the preferred compounds within the scope of the compounds of formula (A) is set forth in PREPARATION 1 and the numerical EXAMPLEs, as well as CHART A. (5R)-5-(methylamino)-5,6-dihydro4H-imidazo[4,5,1-ij]quinoline-2(1H)-thione can alternatively be made from the corresponding non-thio analog, (5R)-(methylamino)-5,6-dihydro-4H-imidao(4,5,1-ij)quinolin-(2H)-one. A method of transforming (5R)-(methylamino)-5,6-dihydro-4H-imidao(4,5,1-ij)quinolin-(2H)-one into (5R)-5-(methylamino)-5,6-dihydro-4H-imidazo[4,5,1-ij]quinoline-2(1H)-thione is set forth in EXAMPLE 8.

Conventional pharmaceutical preparations can be used for the compounds of the invention, e.g. consisting essentially of an inert pharmaceutical carrier and an effective dose of a compound of formula (A) or a pharmaceutically acceptable salt as the active substance. Suitable dosages forms include without limitation plain or coated tablets, capsules, lozenges, powders, solutions, suspensions, emulsions, syrups, suppositories, transdermal patch, etc, with tablet being the preferred dosage form.

The operable neuroprotective amount of the compounds of formula (A) is from about 0.2 to about 8 mg/person/dose. It is preferred that the neuroprotective amount is from about 0.5 thru about 5 mg/person/dose. It is more preferred that the neuroprotective amount is from about 1 to about 3 mg/person/dose. If doses less than this are used the desired effect will not be obtained. If doses greater than this are used, undesirable side effects may occur. The neuroprotective amount of the compounds of the invention when used in accordance with a method of this invention depends on patient condition and the method of administration, and can be adjusted higher or lower by the attending physician depending on patient condition and the observed clinical response to the initial dosage. Treatment in accordance with this invention typically includes one to four daily doses of a compound of the invention. Formulation of a compound of the invention into controlled release dosage forms (either for parenteral or oral use) enables effective once or twice a day dosage protocols.

Compounds of the invention can be administered orally, parenterally, by inhalation spray, topically, rectally, nasally, bucally, vaginally or via an implanted reservoir in a dosage formulation containing conventional non-toxic pharmaceutically acceptable carriers, adjuvants and vehicles. Oral administration is preferred, however parenteral administration may be considered more appropriate/effective where the patient condition is acute. Administration of the compound of the invention is typically continued until patient condition is normalized or until a patient is determine to be no longer susceptible to or disposed to developing or redeveloping the neurodegenerative condition. Dosage administration can be continued using the same or attenuated dosage protocol for prophylaxis of the patient condition.

In another aspect, the present invention provides a neuroprotective pharmaceutical composition comprising a neuroprotective amount of a compound of formula (A) or a pharmaceutically acceptable salt thereof, and a pharmaceutically acceptable carrier therefore. In one embodiment the pharmaceutical composition is prepared in a unit dosage form, for example, a tablet, capsule or caplet for oral dosage form.

In still another aspect, the invention provides a method of manufacturing a pharmaceutical composition useful for preventing neuronal damage or the progression of neuronal damage in a human suffering from or susceptible to such damage. The method comprises the step of preparing a pharmaceutical mixture comprising a compound of formula (A), or a pharmaceutically acceptable salt thereof, and a pharmaceutical acceptable carrier. Portions of the mixture are then used to prepare unit dosage forms containing a neuroprotective amount of a compound of formula (A) or a pharmaceutically acceptable salt.

The amount of a compound of formula (A) or a pharmaceutically acceptable salt used to form the pharmaceutical composition is that amount effective to provide upon delivery by the intended route of administration, a neuroprotective concentration of the compound of formula (A) or a pharmaceutically acceptable salt thereof in the neuronal tissue where protection is desired.

A compound of the invention for use in accordance with this invention can be combined with one or more pharmaceutically acceptable carriers and may be administered, for example, orally in such forms as tablets, capsules, caplets, dispersible powders, granules, lozenges, mucosal patches, sachets, and the like. In such formulations of a compound of the invention is combined with a pharmaceutically acceptable carrier, for example starch, lactose or trehalose, alone or in combination with one or more formulation excipients and pressed into tablets or lozenges or filled into capsules. Optionally, dosage forms intended for oral ingestion administration such as tablets, caplets or capsules can be enterically coated to minimize hydrolysis/degradation in the stomach. In another embodiment the dosage form is formulated for oral administration, and is formed as a prolonged release dosage form using art-recognized formulation techniques for release the of a compound of the invention over a predetermined period of time.

Topical dosage forms, including transdermal patches, intranasally and suppository dosage unit formulations containing a compound of formula (A) or a pharmaceutically acceptable salt thereof and conventional non-toxic pharmaceutically acceptable carriers, adjuvants and vehicles adapted for such routes of administration can also be used in the present neuroprotective method.

The pharmaceutical compositions suitable for injectable use in accordance with this invention include sterile aqueous solutions or dispersions and sterile powders or lyopholysates for the extemporaneous preparation of sterile injectable solutions or dispersions. The dosage forms must be sterile and it must be stable under the conditions of manufacture and storage. The carrier for injectable formulations is typically water but can also include ethanol, a polyol (for example, glycerol, propylene glycol and liquid polyethylene glycol), mixtures thereof, and vegetable oil.

Parenteral dosage forms useful in accordance with the present invention can also be formulated as injectable prolonged release formulations in which a compound of the invention is combined with one or more natural or synthetic biodegradable or biodispersible polymers such as carbohydrates, including starches, gums and esterified or esterified cellulosic derivatives, polyethers, polyesters, polyvinyl alcohols, gelatins, or alginates. Such dosage formulations can be prepared for example in the form of microsphere suspensions, gels, or shaped polymer matrix implants that are well-known in the art for their function as “depot-type” drug delivery systems that provide prolonged release of the biologically active components. Such compositions can be prepared using art-recognized formulation techniques and designed for any of a wide variety of drug release profiles.

Administration of any of the compounds of the invention may include the use of a single compound or a mixture of neuroprotective compounds.

As described in detail in Examples 1 below, assays have been conducted demonstrating the neuroprotective effects that are achieved by the method of the present invention.

### Definitions and Conventions

The definitions and explanations below are for the terms as used throughout this entire document including both the specification and the claims.

### I. Conventions for Formulas and Definitions of Variables

The carbon atom content of variable substituents is indicated in one of two ways. The first method uses a prefix to the entire name of the variable such as “C₁-C₄”, where both “1” and “4” are integers representing the minimum and maximum number of carbon atoms in the variable. The prefix is separated from the variable by a space. For example, “C₁-C₄ alkyl” represents alkyl of 1 through 4 carbon atoms, (including isomeric forms thereof unless an express indication to the contrary is given). Whenever this single prefix is given, the prefix indicates the entire carbon atom content of the variable being defined. Thus C₂-C₄ alkoxycarbonyl describes a group CH₃—(CH₂)ₙ—O—CO— where n is zero, one or two. By the second method the carbon atom content of only each portion of the definition is indicated separately by enclosing the “Cᵢ-Cⱼ” designation in parentheses and placing it immediately (no intervening space) before the portion of the definition being defined. By this optional convention (C₁-C₃)alkoxycarbonyl has the same meaning as C₂-C₄ alkoxy-carbonyl because the “C₁-C₃” refers only to the carbon atom content of the alkoxy group. Similarly while both C₂-C₆ alkoxyalkyl and (C₁-C₃)alkoxy(C₁-C₃)alkyl define alkoxyalkyl groups containing from 2 to 6 carbon atoms, the two definitions differ since the former definition allows either the alkoxy or alkyl portion alone to contain 4 or 5 carbon atoms while the latter definition limits either of these groups to 3 carbon atoms.

### II. Definitions

All temperatures are in degrees Centigrade.

TLC refers to thin-layer chromatography.

HPLC refers to high pressure liquid chromatography.

Saline refers to an aqueous saturated sodium chloride solution.

Chromatography (column and flash chromatography) refers to purification/separation of compounds expressed as (support, eluent). It is understood that the appropriate fractions are pooled and concentrated to give the desired compound(s).

NMR refers to nuclear (proton) magnetic resonance spectroscopy, chemical shifts are reported in ppm (δ) downfield from tetramethylsilane.

CMR refers to C-13 magnetic resonance spectroscopy, chemical shifts are reported in ppm (δ) downfield from TMS.

The terms “nerve cells” and “neurons” or “neuronal cells” are used interchangeably herein to refer to cells in the central nervous system, including the brain.

“Neuronal cells,” “nerve cells,” and “neurons” or “neuronal cells” are used interchangeably herein to refer to those cells that make up the nervous system including, for example, neurons, neural support cells, glia, Schwann cells, cells comprising the vasculature contained within and supplying such cells within the central nervous system including the brain, the brain stem, the spinal cord, and the peripheral nervous system.

“Neuroprotective” or “Neuroprotection” as used in describing and defining the present invention refers to the effect of preventing, arresting, ameliorating, or reducing damage to neuronal cells that leads the death or loss of function of neuronal cells in patients afflicted with conditions known to affect such cells. The term also refers to the capacity or function to protect and/or revive cells which have suffered damage or which are or have been exposed to cell damaging conditions.

“Neuroprotective amount” means the amount of a compound of the invention which is sufficient to be neuroprotective as defined above in patients receiving the treatment.

“Neurodegenerative disorder” is defined here and in the claims as a disorder in which progressive loss of neurons occurs either in the peripheral nervous system or in the central nervous system. Examples of neurodegenerative disorders include: chronic neurodegenerative diseases such as Alzheimer's disease, Parkinson's disease, Huntington's chorea, diabetic peripheral neuropathy, multiple sclerosis, amyotrophic lateral sclerosis; aging; and acute neurodegenerative disorders including: stroke, traumatic brain injury, schizophrenia, peripheral nerve damage, hypoglycemia, spinal cord injury, epilepsy, and anoxia and hypoxia. These examples are not meant to be comprehensive or limiting in any way but serve merely as an illustration of the term “neurodegenerative disorder.”

“(Z)-2-butenedioate” refers to maleate.

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

PREPARATION 1. (R)-Naproxen chloride

R-naproxen (260 g), methylene chloride (3.33 kg) and DMF (8.2 ml) are added to a reactor. Oxalyl chloride (191.8 g) is slowly added to this mixture. After addition of the oxalyl chloride, the slurry is stirred at 5 to 100 and then slowly warmed to 20-25°. The resulting mixture is concentrated to remove the methylene chloride, branched octane is added to the concentrate and the mixture is again concentrated. More branched octane is added to the concentrate and the mixture is cooled to 0° and stirred to crystallize. The crystal slurry is filtered, the crystal cake is washed with octane and dried at 20-25° to obtain the title compound.

The filtrate from the first crop is concentrated, branched octane is added and the mixture is cooled and stirred to obtain a second crop of the title compound. The slurry is filtered, the crystal cake is washed with branched octane and dried at 20-25°.

### Example 1

1-Benzyl-4H-imidazo[4,5,1-ij]quinolin-2(1H)-one (II)

A mixture of 4H-imidazo[4,5,1-ij]quinolin-2(1H)-one (I, J. Heterocyclic Chem.,19, 837-49 (1982), 1.0g, 5.8mmol) in DMF (10 ml) is cooled to 0° and treated with potassium t-butoxide in THF (1.98 M, 3.2 ml, 6.3 mmol) maintaining the reaction temperature at 0°. The resulting mixture is stirred at 0° for 10 minutes. Benzyl bromide (0.73 ml, 6.1 mmol) is then added while maintaining the reaction temperature at 0°. After 1 hr, the mixture is partitioned with methyl t-butyl ether (MTBE) from water followed by several water washes. The MTBE phase is concentrated under reduced pressure. The concentrate is cooled to 0°, filtered and washed two times with 0° MTBE. The product is dried at 50° under reduced pressure with a nitrogen purge to give the title compound, CMR (CDCl₃, 100 MHz) L 153.78, 136.44, 128.69, 127.67, 127.60, 126.73, 125.86, 122.90, 122.78, 121.28, 116.92, 116.17, 108.36, 44.95 and 42.37.

### Example 2

(5R*,6R*)-1-benzyl-5-bromo-6-hydroxy-5,6-dihydro-4H-imidazo[4,5,1-ij]quinolin-2(1H)-one (III)1-Benzyl-4H-imidazo[4,5,1-ij]quinolin-2(1H)-one (II, EXAMPLE 1, 240 g), acetonitrile (1.086 kg), water (227 ml) and fluoboric acid (48.5%, 13.4 g) are mixed and cooled to 0 to 5°. Dibromantin (163.5 g) is slurried into acetonitrile and is added to the reaction mixture. The reaction is carried out for about 3 hr at 0 to 5°. After the reaction is complete, methyl t-butyl ether is added over about 45 minutes keeping the reaction temperature in the pot below 10°. The slurry is cooled to −10 to −15°, stirred for an hour and then filtered. The product is washed with precooled methyl t-butyl ether, dried with 40° nitrogen to give the title compound, CMR (CDCl₃) δ 156.0, 137.8, 130.5, 129.6, 129.3, 129.1, 126.6, 123.6, 122.5, 119.6, 110.4, 69.9, 49.6, 47.7, 46.9 and 43.8.

### Example 3

(5S,6S)-1-Benzyl-5-bromo-2-oxo-1,2,5,6-tetrahydro-4H-imidazo[4,5,1-ij]quinolin-6-yl (2R)-2-(6-methoxy-2-naphthyl)propanoate (IVA) and (5R,6R)-1-benzyl-5-bromo-2-oxo-1,2,5,6-tetrahydro-4H-imidazo[4,5,1-ij]quinolin-6-yl (2R)-2-(6-methoxy-2-naphthyl)propanoate (IVB)(5R,6R)-1-Benzyl-5-bromo-6-hydroxy-5,6-dihydro-4H-imidazo[4,5,1-ij]quinolin-2(1H)-one (III, EXAMPLE 2, 143 g), methylene chloride (3,136 g), N-methyl morpholine (100.2 g) and 4-dimethylaminopyridine (497 mg) are added to the reactor and the mixture is cooled to 0 to 5°. (R)-Naproxen chloride (PREPARATION 1, 118.5 g) dissolved in methylene chloride (694 ml) is added to the reactor over about 1 hr and the mixture is stirred at 0 to 5° to complete the reaction. If necessary, additional naproxen chloride is added to complete the reaction. Potassium carbonate solution diluted with water is added to the mixture. The aqueous phase is extracted with methylene chloride and the combined methylene phase is washed with water. The washed mixture is concentrated by vacuum distillation and solvent exchange with ethyl acetate is performed. The concentrate is cooled to −10° and stirred. The crystal slurry is filtered and the crystal cake is washed with precooled methyl t-butyl ether and dried at 50° to give the title compound in solid form, (5S,6S)-1-benzyl-5-bromo-2-oxo-1,2,5,6-tetrahydro-4H-imidazo[4,5,1-ij]quinolin-6-yl (2R)-2-(6-methoxy-2-naphthyl)propanoate (IVA), CMR (CDCl₃) δ 173.2, 157.8, 153.4, 136.1, 134.6, 133.7, 129.2, 128.8, 127.8, 127.8, 127.6, 127.2, 125.9, 125.9, 125.6, 121.5, 121.4, 119.1, 113.2, 109.0, 105., 105.6, 69.2, 55.3, 45.4, 45.2, 42.5, 41.7 and 18.3.

The undesired isomer, (5R,6R)-1-benzyl-5-bromo-2-oxo-1,2,5,6-tetrahydro-4H-imidazo[4,5,1-ij]quinolin-6-yl (2R)-2-(6-methoxy-2-naphthyl)propanoate (IVB) is in the filtrate and can be recovered by means well known to those skilled in the art, (5R,6R)-1-benzyl-5-hydroxy-6-(methylamino)-5,6-dihydro-4H-imidazo[4,5,1-ij]quinolin-2(1H)-one, CMR 173.2, 157.9, 153.4, 136.1, 135.0, 133.8, 129.2, 128.9, 128.8, 127.8, 127.6, 127.4, 125.8, 125.8, 125.7, 121.6, 121.5, 119.3, 113.1, 109.1, 105.7,68.7, 55.3, 45.3, 45.2, 42.2, 41.3 and 18.1 δ.

### Example 4

(5R,6R)-1-benzyl-5-hydroxy-6-(methylamino)-5,6-dihydro-4H-imidazo[4,5,1-ij]quinolin-2(1H)-one (V)(S,6S)-1-Benzyl-5-bromo-2-oxo-1,2,5,6-tetrahydro-4H-imidazo[4,5,1-ij]quinolin-6-yl (2R)-2-(6-methoxy-2-naphthyl)propanoate (IVA, EXAMPLE 3, 110 g) is slurried in acetonitrile (1,297 g). After adding aqueous methylamine (40 wt %, 327 g) the reaction is carried out for about 12 hr at about 30°. After the reaction is complete, the mixture is concentrated and ethyl acetate is added. Dilute hydrochloric acid is added to make the water-soluble salt of the title compound. The byproduct (R-naproxen acetamide impurity) is insoluble in water and stays in the ethyl acetate phase. Further extractions and washes are carried out for better separation of the (naproxen acetamide) impurity with minimum loss of the desired product. Then a sodium hydroxide solution is added to the aqueous phase and the hydrochloride salt of the title compound is converted to the free base. The free base is less soluble in water and is extracted into ethyl acetate. The product mixture is concentrated and solvent exchanged with ethyl acetate to remove water. Crystallization is performed by adding branched chain octane and cooling the mixture. The resulting slurry is filtered, washed and dried at 50° to give the title compound, CMR (CDCl₃) δ 153.7, 136.3, 128.7, 127.8, 127.7, 125.7, 121.3, 119.9, 118.6, 107.5, 66.2, 60.1, 45.1, 42.6 and 34.0.

### Example 5

(7aS,8aR)-4-benzyl-8-methyl-7,7a,8,8a-tetrahydroazireno[2,3-c]imidazo[4,5,1-ij]quinolin-5(4H)-one (VI)(5R,6R)-1-benzyl-5-hydroxy-6-(methylamino)-5,6-dihydro-4H-imidazo[4,5,1-ij]quinolin-2(1H)-one (V, EXAMPLE 4, 70 g) and THF (1,389 g) is concentrated to remove any moisture with distillate as a precaution due to reactivity of n-butyl lithium towards water. The mixture is cooled to about −10° and n-butyllithium is added to make the lithium salt of the starting material with formation of n-butane byproduct in an exothermic reaction. Benzene sulfonyl chloride is added slowly to make benzene sulfonate in an exothermic reaction. The reaction mixture is warmed to 20-25° to complete the reaction. Agueous potassium carbonate solution is added to scavenge the benzene sulfonic acid and the mixture is stirred to allow crystallization. Water is added to complete crystallization, the slurry is stirred, cooled and filtered. The crystal cake is washed with water followed by branched chain octane and dried at 40 to 50° to give the title compound, CMR (CDCl₃) δ 154.1, 136.3, 128.6, 127.9, 127.6, 124.3, 120.7, 119.7, 107.4, 46.7, 44.9, 40.7, 38.1 and 37.6.

### Example 6

(5R)-(methylamino)-5,6-dihydro-4H-imidazo[4,5,1-ij]quinolin-2(1H)-one (VII).

A mixture of (7aS,8aR)-4-benzyl-8-methyl-7,7a,8,8a-tetrahydroazireno[2,3-c]imidazo[4,5,1-ij]quinolin-5(4H)-one (VI, EXAMPLE 5, 40 g) t-amyl alcohol (42.4 g) and anhydrous ammonia (1,200 g) is treated with lithium at −33°. After the lithium addition is complete, the reaction mixture changes from a yellow slurry to a dark blue mixture. This dark blue mixture is stirred for 30-60 minutes and then quenched with the addition of water. The cooling is removed from the condenser and the ammonia is allowed to evaporate. The residue is dissolved in methanol. This mixture is then concentrated to dryness to give the title compound, which is carried on directly to the next step without isolation.

### Example 7

(5R)-(methylamino)-5,6-dihydro-4H-imidazo[4,5,1-ij]quinolin-2(1H)-one (Z)-2-butenedioate (1:1) (VIII)(5R)-(methylamino)-5,6-dihydro-4H-imidazo[4,5,1-ij]quinolin-2(1H)-one (VII, EXAMPLE 6, 28.0 g) is dissolved in water and the pH is adjusted to 10 with the addition of hydrochloric acid. The mixture is applied in portions to an XAD-16 resin column which is eluted first with water and then with ethanol. The inorganic salts are eluted from the column first with the desired product eluted with the ethanol. The ethanol eluate from the column is treated with maleic acid and the water level is lowered through azeotropic distillation of the ethanol. The precipitated product is isolated by filtration, rinsed with ethyl acetate and dried to give the title compound, CMR (DMSO-d₆) δ 167.6, 153.9, 136.4, 127.1, 121.5, 119.6, 114.1, 107.5, 51.9, 31.3 and 26.5.

### Example 8

(5R)-5-(Methylamino)-5,6-dihydro-4H-imidazo[4,5,1-ij]quinoline-2(1H)-thione.

A mixture of (5R)-(methylamino)-5,6-dihydro-4H-imidazo[4,5,1-ij]quinolin-2(1H)-one (VII, EXAMPLE 6, 15.0 g, 73.8 mmol) and tetraphosphorus decasulfide (36.1 g, 81.2 mmol) in pyridine (300 mL) is heated in a 125° oil bath under nitrogen. The reaction is stirred for 5 hr. The mixture is cooled to 20-25° and the pyridine is removed under reduced pressure. Sodium hydroxide (2.2 N, 200 mL) is added. Sodium hydroxide (1 N) is then added. The mixture is saturated with sodium chloride and extracted with methylene chloride (2.5 L, in portions). The organic phase is absorbed onto silica gel (40 g) and purified via column chromatography (silica gel; 225 g; methanol/methylene chloride, 3.5-5.0/96.5-95) to give a solid. Recrystallization of this material from methanol/ethyl acetate/hexanes give the title compound, mp=210-213°; IR (drift) 2940, 2907, 2884, 1483, 1458, 1391, 1366, 1354, 1254, 1239, 1229, 895, 762, 734, 630 cm⁻¹; NMR (300 MHz, CDCl₃) 7.12, 7.03, 7.00, 4.30, 3.96, 3.30-3.50, 3.15, 2.88 and 2.57 δ; MS (EI, m/z) 219 (M⁺), 190, 189, 187, 186, 164, 163, 155, 145; HRMS (FAB) calculated for C₁₁H₁₃N₃S (MH⁺)=220.0908, found 220.0904.

### Example 9

(5R)-5-(Methylamino)-5,6-dihydro-4H-imidazo[4,5,1-ij]quinoline-2(1H)-thione malate.

A mixture of maleic acid (0.317 g, 2.36 mmol) in a minimal amount of methanol (˜1 mL) is added to a mixture of (5R)-5-(methylamino)-5,6-dihydro-4H-imidazo[4,5,1-ij]quinoline-2(1H)-thione (EXAMPLE 8, 0.493 g, 2.25 mmol) in methylene chloride. The resulting solid is collected by filtration to give the title compound, mp=195-196°; [α]²⁵D=−60° (c 0.93, methanol); IR (drift) 3140, 3112, 3060, 2969, 1627, 1619, 1568, 1481, 1455, 1398, 1389, 1361, 1220, 868 and 747 cm⁻¹; NMR (300 MHz, CD₃OD) 7.20-7.30, 7.10-7.20, 6.26, 4.49, 4.31, 4.05-4.20, 3.47, 3.28 and 2.83 δ; CMR (100 MHz, DMSO-d₆+CD₃OD) 170.4, 169.4, 136.6, 131.1, 130.9, 125.1, 122.1, 116.2, 109.6, 53.9, 43.1, 31.9 and 27.2 δ; MS (ESI, m/z) 220.1 (MH⁺).

### Example 10

Test for Neuroprotective Property.

To demonstrate the neuroprotective properties of the compounds of the invention, in vovo studies were conducted in an animal model of neurotoxicity known in the art.

Experimental Procedure:

3-acetylpyridine (3-AP), a nicotinamide antagonist and a potent rat neurotoxin, was administered to groups of rats.

Sumanirole (1-20 mg/kg, PO) was given either pre- or post 3-AP treatment and animals were sacrificed 96 hours later. Neuronal cell counts were performed in the inferior olive and cGMP, ATP and rotorod performance were used as surrogate toxicity markers.

Results:

3-AP treatment produced significant decreases in cerebellar cGMP and ATP, decrements in rotorod performance and a significant decrease in inferior olive neurons. Sumanirole, given either before or after 3-AP, significantly attenuated 3-AP induced reductions in cGMP, ATP and rotorod performance in a dose-related manner. Sumanirole also significantly reduced the inferior olive neuronal cell loss produced by 3-AP. Pretreatment with raclopride did not block the neuroprotective effects of sumanirole.

Summary:

The data demonstrate that sumanirole has in vivo neuroprotective properties and such properties do not appear to be related to the compound's D2 agonist properties.

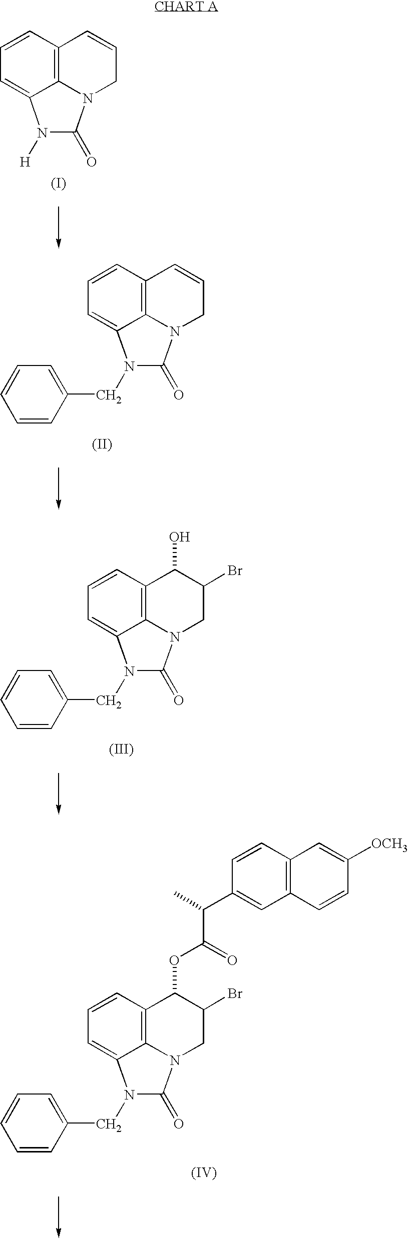

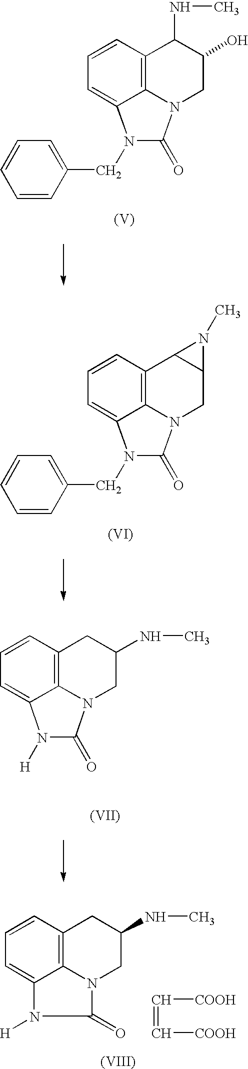

1. A method of treating a human suffering from, or susceptible to, a disease condition known to result in, or from, loss of neuronal cells or loss of neuronal cell function by reducing loss of neuronal cells or neuronal cell function resulting from such disease condition, said method comprising the step of administering to said human a neuroprotective amount of a compound of formula (A) 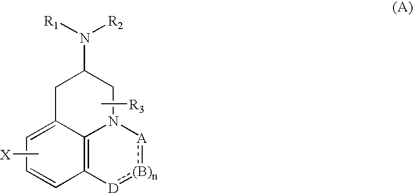
or a pharmaceutically acceptable salt, wherein in formula (A),
R₁, R₂ and R₃ are the same or different and are:
—H,
C₁-C₆ alkyl,
C₃-C₅ alkenyl,
C₃-C₅ alkynyl,
C₃-C₅ cycloalkyl,
C₄-C₁₀ cycloalkyl,
phenyl substituted C₁-C₆ alkyl,
—NR₁R₂ where R₁ and R₂ are cyclized with the attached nitrogen atom to produce pyrrolidiyl, piperidinyl, morphoninyl, 4-methyl piperazinyl or imidazolyl;
X is:
—H,
C₁-C₆ alkyl,
—F, —Cl, —Br, —I,
—OH,
C₁-C₆ alkoxy,
cyano,
carboxamide,
carboxyl,
(C₁-C₆ alkoxy)carbonyl,
A is:
CH,
CH₂,
CH-(halogen) where halogen is —F, —Cl, —Br, —I,
CHCH₃,
C═O,
C═S,
C—SCH₃,
C═NH,
C—NH₂,
C—NHCH₃,
C—NHCOOCH₃,
C—NHCN,
SO₂,
N;
B is:
CH₂,
CH,
CH-(halogen) where halogen is as defined above,
C═O,
N,
NH,
N—CH₃,
D is:
CH,
CH₂,
CH-(halogen) where halogen is as defined above,
C═O,
O,
N,
NH,
N—CH₃;
and n is 0 or 1, and where [symbol] is a single or double bond, with the provisos:
(1) that when n is 0, and
A is CH₂, CH-(halogen) where halogen is as defined above, CHCH₃, C═O, C═S, C═NH, SO₂;
then D is CH₂, CH-(halogen) where halogen is as defined above, C═O, O, NH, N—CH₃;
(2) that when n is 0, and
A is CH, C—SCH₃, C—NH₂, C—NHCH₃, C—NHCOOCH₃, C—NHCN, N; then
D is CH, N;
(3) that when n is 1, and
A is CH₂, CH-(halogen) where halogen is as defined above, CHCH₃, C═O, C═S, C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N; and pharmaceutically acceptable salts thereof to the human.

2. A method for preventing neuronal damage or the progression of neuronal damage in a patient suffering from or susceptible to such neuronal damage, said method comprising administering to the patient a neuroprotective amount of a compound of formula (A) 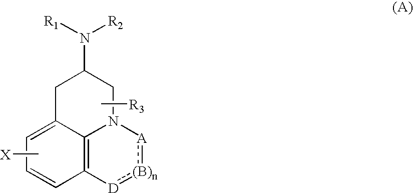
or a pharmaceutically acceptable salt, wherein in formula (A),
R₁, R₂ and R₃ are the same or different and are:
—H,
C₁-C₆ alkyl,
C₃-C₅ alkenyl,
C₃-C₅ alkynyl,
C₃-C₅ cycloalkyl,
C₄-C₁₀ cycloalkyl,
phenyl substituted C₁-C₆ alkyl,
—NR₁R₂ where R₁ and R₂ are cyclized with the attached nitrogen atom to produce pyrrolidiyl, piperidinyl, morphoninyl, 4-methyl piperazinyl or imidazolyl;
X is:
—H,
C₁-C₆ alkyl,
—F, —Cl, —Br, —I,
—OH,
C₁-C₆ alkoxy,
cyano,
carboxamide,
carboxyl,
(C₁-C₆ alkoxy)carbonyl,
A is:
CH,
CH₂,
CH-(halogen) where halogen is —F, —Cl, —Br, —I,
CHCH₃,
C═O,
C═S,
C—SCH₃,
C═NH,
C—NH₂,
C—NHCH₃,
C—NHCOOCH₃,
C—NHCN,
SO₂,
N;
B is:
CH₂,
CH,
CH-(halogen) where halogen is as defined above,
C═O,
N,
NH,
N—CH₃,
D is:
CH,
CH₂,
CH-(halogen) where halogen is as defined above,
C═0,
O,
N,
NH,
N—CH₃;
and n is 0 or 1, and where [symbol] is a single or double bond, with the provisos:
(1) that when n is 0, and
A is CH₂, CH-(halogen) where halogen is as defined above, CHCH₃, C═O, C═S, C═NH, SO₂;
then D is CH₂, CH-(halogen) where halogen is as defined above, C═O, O, NH, N—CH₃;
(2) that when n is 0, and
A is CH, C—SCH₃, C—NH₂, C—NHCH₃, C—NHCOOCH₃, C—NHCN, N; then
D is CH, N;
(3) that when n is 1, and
A is CH₂, CH-(halogen) where halogen is as defined above, CHCH₃, C═O, C═S, C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N; and pharmaceutically acceptable salts thereof to the human.

3. A method according to claim 1 or 2 wherein the disease condition is selected from Parkinson's disease, primary neurodegenerative disease; Huntington's Chorea; stroke and other hypoxic or ischemic processes; neurotrauma; metabolically induced neurological damage; sequelae from cerebral seizures; hemorrhagic stroke; secondary neurodegenerative disease (metabolic or toxic); Alzheimer's disease, other memory disorders; or vascular dementia, multi-infarct dementia, Lewy body dementia, or neurogenerative dementia.

4. A method according to claim 3 wherein the disease condition is Parkinson's disease.

5. A method according to claim 1 or 2 wherein the compound of formula (A) is administered orally, intra-nasally, buccally, intra-pulmonary, parenterally and rectally.

6. A method according to claim 5 wherein the compound of formula (A) is administered orally.

7. A method according to claim 1 or 2 wherein the neuroprotective amount is from about 0.2 to about 8 mg/person/dose.

8. A method according to claim 7 where the neuroprotective amount is from about 0.5 to about 5 mg/person/dose.

9. A method according to claim 8 wherein the neuroprotective amount is from about 1 to about 3 mg/person/dose.

10. A method according to claim 1 or 2 wherein the pharmaceutically acceptable salt is selected from the group consisting of salts of the following acids methanesulfonic, hydrochloric, hydrobromic, sulfuric, phosphoric, nitric, benzoic, citric, tartaric, fumaric, maleic, CH₃—(CH₂)ₙ—COOH where n is 0 thru 4, HOOC—(CH₂)_{N}—COOH where n is as defined above.

11. A method according to claim 1 or 2 wherein the compound of formula (A) is (5R)-(methylamino)-5,6-dihydro-4H-imidazo[4,5,1-ij]quinolin-2(1H)-one or a pharmaceutically acceptable salt thereof.

12. A method according to claim 11 where the pharmaceutically acceptable salt of (5R)-(methylamino)-5,6-dihydro-4H-imidazo[4,5,1-ij]quinolin-2(1H)-one is (5R)-(methylamino)-5,6-dihydro-4H-imidazo[4,5,1-ij]quinolin-2(1H)-one (Z)-2-butenedioate (1:1).

13. A method according to claim 1 or 2 where the compound of formula (A) is (5R)-(methylamino)-5,6-dihydro-4H-imidazo[4,5,1-ij]quinolin-2(1H)-thione or a pharmaceutically acceptable salt thereof.

14. A method according to claim 13 where the pharmaceutically acceptable salt of (5R)-(methylamino)-5,6-dihydro-4H-imidazo[4,5,1-ij]quinolin-2(1H)-thione is (5R)-(methylamino)-5,6-dihydro-4H-imidazo[4,5,1-ij]quinolin-2(1H)-thione (Z)-2-butenedioate (1:1).
